(19) United States
(12) Patent Application Publication
(10) Pub. No.: US 2013/0102532 A1
(43) Pub. Date: Apr. 25, 2013

(54) HB-EGF Composition and Use Thereof to Treat a Condition Associated With Enhanced Urothelium Permeability

(71) Applicants: Robert Uger, Ontario (CA); Penka Petrova, Toronto (CA)

(72) Inventors: Robert Uger, Ontario (CA); Penka Petrova, Toronto (CA)

(73) Assignee: TRILLIUM THERAPEUTICS INC., Toronto, ON (CA)

(21) Appl. No.: 13/641,478

(22) PCT Filed: Apr. 12, 2011
(86) PCT No.: PCT/CA11/00377

§ 371 Date: Dec. 18, 2012

Related U.S. Application Data
(60) Provisional application No. 61/326,291, filed on Apr. 21, 2010.

(51) Int. Cl. A61K 38/18

(52) U.S. Cl. CPC A61K 38/1808
USPC 514/9.6

(57) ABSTRACT

HB-EGF is exploited to treat conditions associated with enhanced urothelium permeability, including interstitial cystitis.

### FIELD

This disclosure relates to the protein known as heparin-binding epidermal growth factor-like growth factor (HB-EGF). It relates more particularly to medically useful compositions that comprise HB-EGF, and the use of those compositions to repair urothelium damage associated with various medical conditions that include interstitial cystitis.

### BACKGROUND

Heparin-binding epidermal growth factor-like growth factor, or HB-EGF, is a member of the EGF protein family that also includes EGF, TGFα, amphiregulin and betacellulin. HB-EGF itself was originally identified in 1990 as a macrophage-secreted, heparin binding growth factor. Like other members in the EGF family, HB-EGF exerts its biological effects through binding to the EGF receptor. However, unlike most members of the EGF family, HB-EGF binds heparin with high affinity, which appears to potentiate binding to the EGF receptor (her-1, ErbB1). HB-EGF also binds the receptor her-4 (ErbB4) and nardilysin, which makes it unique within the EGF family.

Cloning of the cDNA encoding human HB-EGF was reported in 1991 (Higashiyama et al, Science, 251:936). The mature form of HB-EGF is a secreted protein that is processed from a 208 amino acid transmembrane precursor known as pro-HB-EGF. Mature HB-EGF comprises the 86 amino acids that span residues 63-148 of the precursor represented herein by SEQ ID NO:1. Numerous microheterogeneous forms of HB-EGF also are known, and include different N-terminal truncations of the precursor form, including residues 63-148, residues 73-148, residues 77-148 and residues 82-148. One particularly active form of human HB-EGF comprises residues 74-148 (SEQ ID NO:2). Production of these proteins as recombinant products is described in U.S. Pat. No. 5,811,393 issued Sep. 22, 1998.

The use of HB-EGF to treat various medical indications has been proposed. In U.S. Pat. No. 7,276,479 issued Oct. 2, 2007, Besner and Pillai describe the use of HB-EGF to treat conditions associated with intestinal ischemia, such as necrotizing enterocolitis, shock, sepsis and intestinal angina, in both pediatric and adult patients. In U.S. Pat. No. 6,232,289 issued May 15, 2001, Keay et al propose the use of HB-EGF to inhibit a urine-borne antiproliferative factor (APF) that is elevated in patients with interstitial cystitis, a condition marked in some patients by erosion of the bladder urothelium which has an inflammatory component and is marked by pain, as well as frequency and urgency of urination in the afflicted subject.

The precise cause of interstitial cystitis (IC) is not known, but proposed etiologies include infection, allergic or immune disorders, endocrine disturbance, toxic urinary chemicals, defective transitional mucosa, psychiatric disorders, neurogenic disorders, and lymphatic or vascular obstruction. Proposed treatments include pentosan polysulfate, glycosaminoglycans such as hyaluronic acid (see U.S. Pat. No. 5,880,108) and chondroitin sulfate (see U.S. Pat. No. 6,083,933, and U.S. Pat. No. 7,772,210), anti-inflammatory or immunosuppressant therapy, muscle relaxants, anti-histamines, and analgesics. Of these, the FDA has approved only pentosan polysulfate that is delivered orally, and a 50% solution of dimethylsulfoxide, which is delivered to the bladder by intravesical instillation.

Whatever the cause, interstitial cystitis is a disease of the urothelium. Because the major roles of the urothelium are (1) to serve as a barrier in preventing bacterial and crystal adherence, and (2) to prevent penetration of urinary solutes into the bladder wall, loss of this barrier function, as occurs in IC, certainly plays a role in the pathophysiology of this disease (see Hurst et al, April 2007, Urology, 69 (supplement 4A): 17-23). It would thus be desirable to provide a treatment effective to promote the growth and barrier properties of the urothelium, for use therapeutically in subjects suffering from cystitis and other conditions associated with damaged urothelium.

### SUMMARY

It has now been determined that extensive proliferative changes occur, resulting in thickening of both urothelium and underlying bladder tissue layers, when HB-EGF is administered to the bladder by instillation. Moreover, administration of HB-EGF has the dual effect, and further medical benefit, of reducing permeability of uroepithelial cells and tissue, thus promoting the barrier function required to reduce solute sensitivity in subjects having damaged urothelium, as in patients with cystitis. This HB-EGF effect is particularly surprising in that the EGFR, the receptor for HB-EGF, is not present in the umbrella cell layer in normal bladder. The present disclosure thus relates to the use of HB-EGF in amounts effective to reduce permeability of urothelium, particularly to treat subjects presenting with damaged urothelium, including subjects presenting with cystitis and especially interstitial cystitis.

Thus, in accordance with one aspect of the present disclosure, there is provided a method for treating a subject to reduce permeability of damaged urothelium, comprising delivering to the urothelium of the subject an effective amount of HB-EGF. In related aspects, the method is performed to treat subjects presenting with cystitis, including interstitial cystitis and related conditions associated with damaged urothelium. Similarly, the present disclosure provides for the use of HB-EGF in the preparation of a medicament to reduce urothelium permeability in a subject in need thereof, including a subject presenting with interstitial cystitis. The disclosure further provides for the use of HB-EGF to reduce urothelium permeability in a subject in need thereof, including a subject presenting with interstitial cystitis.

In a related aspect of the disclosure, there is provided a composition comprising HB-EGF in a unit dose effective to reduce permeability of urothelium in a subject in need thereof. In embodiments, the composition comprises HB-EGF in a unit dose within the range from 10 mgs to 1,000 mgs, such as 50-200 mgs.

In a further related aspect, the present disclosure provides a kit comprising HB-EGF in a unit dose effective, when delivered by instillation to the bladder of a subject in need thereof, to reduce permeability of the urothelium, and instruction for the use thereof to effect such treatment. In embodiments, the kit further comprises an aqueous carrier suitable for reconstituting the HB-EGF into a dosage form suitable for intravesical delivery, and optionally, a catheter for instilling the composition.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

These and other aspects of the disclosure are now described in greater detail with reference to the accompanying drawings in which:

FIG. 1 shows the effect of HB-EGF in vivo on proliferation of bladder cells in normal mice; and

FIG. 2 shows the effect of HB-EGF in vivo on permeability of acid-damaged mouse bladder.

### DETAILED DESCRIPTION

The disclosure relates to the use of heparin-binding epidermal growth factor-like growth factor (HB-EGF) to promote thickening and/or to reduce permeability of urothelium of the bladder and other tissues, as a means for treating subjects that present with a medical condition or disorder marked by urothelium damage. Such conditions include principally those associated with damaged, eroded or inflamed urothelium, including particularly those that are associated with enhanced urothelial permeability.

An HB-EGF protein is a protein species that has HB-EGF activity and includes at least amino acids 82-148 of SEQ ID NO:1, or a functional equivalent thereof which incorporates one, two or three amino acid additions, substitutions such as conservative amino acid substitutions, or deletions, and includes naturally existing HB-EGF variants that retain HB-EGF activity. HB-EGF activity of a particular protein is confirmed when, for instance, that protein tests positive in a cell proliferation assay using Balb/c 3T3 cells.

In embodiments, the HB-EGF protein comprises amino acids 77-148, or amino acids 73-148, or amino acids 63-148 of SEQ ID NO:1. In a specific, preferred embodiment, the HB-EGF protein is human HB-EGF comprising amino acids 74-148 (SEQ ID NO:2). This HB-EGF species is believed to be the main, endogenous species in humans. It will be appreciated that any of the other microheterogeneous forms of human HB-EGF having HB-EGF activity can also be used in the present disclosure. Also useful are HB-EGF-active variants of these forms, which variants incorporate 1, 2 or 3 or more amino acid additions, deletions or substitutions, where substitutions are desirably conservative amino acid substitutions and additions and deletions desirably are N-and/or C-terminal additions or deletions. A variant HB-EGF protein also desirably retains heparin binding activity.

The HB-EGF product can be extracted from natural sources, such as cell lines including U-937 (ATCC CRL 1593), and can be chemically synthesized, but is produced preferably as a recombinant product by culturing a host organism that has been engineered genetically to express an HB-EGF-encoding gene from a suitable promoter, as described in greater detail in U.S. Pat. No. 5,811,393. HB-EGF is also now commercially available as a recombinant product of bacterial (E. coli) expression. In the alternative, HB-EGF is produced in a eukaryotic host such as yeast, filamentous fungi, or mammalian cells such as CHO or COS, so that the expression product is glycosylated, particularly at residues 75 and 85 as in the native protein.

It will further be appreciated that the HB-EGF protein can be provided and used as a protein conjugate in which HB-EGF is conjugated, either covalently or by physical association, with a carrier molecule useful for any intended purpose. The carrier molecule may be an Fc region of an IgG to provide a dimeric form of HB-EGF, or a polymer such as a polyethylene glycol, serum albumin, amylose, or the like useful to extend or slow the release of active drug, or another protein or molecule having utility in treating the condition.

The HB-EGF is useful to treat subjects presenting with medical conditions that feature or is associated with damage to the urothelium that manifests as a thinning of the urothelium and/or in enhanced permeability thereof, such as erosion or inflammation of the urothelium. The urothelium is the tissue layer that lines much of the urinary tract, including the renal pelvis, the ureters, the bladder and part of the urethra, and protects underlying tissues against noxious urine components that include soluble irritants such as K+, while also stretching to accommodate urine volume pressures.

Subjects that would benefit from treatment with HB-EGF can be revealed using tests established for diagnosing patients with interstitial cystitis and related bladder disorders. Cystoscopy allows the urologist to look into the bladder and carry out a number of tests and is a standard investigation in urology. A narrow tube is inserted into the bladder via the urethra. It has two or more channels: one carrying an endoscope permitting visual examination of the inside of the bladder, and the other carrying fluid for instillation into the bladder. Cystoscopy can be performed either in the physician's office using local anaesthesia but without hydrodistension (stretching the bladder), or in a clinic under general or spinal anaesthesia with hydrodistension. The office cystoscopy with local anaesthesia is an investigation to exclude the possibility of other causes of the symptoms, such as tumours, stones, etc. Cystoscopy also makes it possible to detect any scarring or thinning of the bladder wall which might be Hunner's ulcer/lesion, and to detect glomerulations, which are pinpoint petechial haemorrhages seen in around 90% of IC patients. Cystoscopy under general or spinal anaesthesia is performed when IC or related urothelium thinning is suspected in order to carry out hydrodistension in which the bladder is filled with fluid twice, the first time to maximum capacity to assess bladder capacity under anaesthesia, the second time less in order to inspect the bladder wall.

The Hunner's lesion typically presents as a circumscript, reddened mucosal area with small vessels radiating towards a central scar, with a fibrin deposit or coagulum attached to this area. This site ruptures with increasing bladder distension, with petechial oozing of blood from the lesion and the mucosal margins in a waterfall manner. A slightly bullous edema develops post-distension with varying peripheral extension.

As well, a bladder biopsy may be carried out. This involves taking a minimum of three small samples of tissue from different levels in the bladder wall, including from the detrusor muscle, at several different sites in the bladder. These samples are then examined microscopically by the pathologist and may reveal an increase in mast cells in the detrusor muscle in the bladder wall. Mast cells play a role in allergic and inflammatory reactions in the body's tissues. They can degranulate and release histamine. Mast cell counts are often higher in IC patients than in patients with other bladder diseases. A bladder biopsy can also be examined by a pathologist to determine urothelium thickness.

Subject candidates can also be assessed using the potassium sensitivity test to identify those having a leaky bladder, i.e., a bladder having damage that allows sensitivity to instilled potassium, as described below.

Diseases that can be targeted and treated using HB-EGF include infectious diseases that afflict epithelia where diverse microbes (viruses, bacteria, fungi) have surface structures that bind specific features of particular epithelial cells. One common infectious disease is urinary tract infection (UTI). UTIs afflict approximately half of all women during their lifetime, and about 25% of these women will suffer recurrent UTIs. The majority of these infections are due to uropathogenic E. coli. However, UTIs can also develop in healthcare settings and such infections are caused by a greater frequency of non-E. coli bacteria.

Another condition which affects the urothelium is interstitial cystitis (IC), a condition with symptoms similar to UTI (frequency, urgency, pressure and/or pain). Urine culture, however, is negative. During hydrodistention of the bladder, small petechial hemorrhages (aka glomerulations) are frequently found throughout the bladder. Larger “Hunner's Ulcers”, known for their characteristic waterfall bleeding effect, represent larger areas of bladder wall thinning and/or trauma. The cause of IC is currently unknown though some suggest that it could be genetic, the result of traumatic injury (chemical exposure), infection, or autoimmune disease. The term “interstitial cystitis” is used interchangeably with such other terms as “painful bladder syndrome” (PBS), “bladder pain syndrome” (BPS), and “hypersensitive bladder syndrome” (HBS).

Thus, HB-EGF is useful to treat a subject presenting with a condition marked by damaged urothelium, particularly damaged bladder urothelium, including but not limited to urinary tract infection having associated damaged bladder urothelium such as permeable bladder epithelium, and interstitial cystitis having associated damaged bladder urothelium such as permeable bladder epithelium, as well as Hunner's lesions/ulcers, and related conditions including hemorrhagic cystitis, radiation-induced cystitis, acute bacterial cystitis, radiation cystitis, chronic pelvic pain, urethral syndrome, overactive bladder, and prostatitis.

Conditions that are “related” can be revealed in a given subject using the so-called potassium sensitivity test (PST), in which a 3% KCl solution is instilled into the bladder of the subject. A response that includes sensation of pain or urgency indicates the subject has a damaged urothelium resulting in enhanced permeability, and is a candidate for treatment in accordance with the present method. As well, subjects that present with bladder lining damage visible either by cystoscopy or biopsy also are candidates for the present treatment.

In one embodiment, subjects for treatment are those presenting with substantially normal urine levels of the antiproliferative factor (APF) identified by Keay et al in for instance in U.S. Pat. No. 6,376,197 published Apr. 23, 2002. In another embodiment, subjects for treatment are those presenting with elevated urine levels of APF. In a related embodiment, subjects selected for treatment with the present HB-EGF composition are not assessed for endogenous APF level, and HB-EGF administration proceeds without this step in patient recruitment.

The subject is treated using a dosing regimen that is most appropriate for the given condition, and delivers the drug for desired effect. The desired effect can be revealed as a reduction in the blood level of any marker that should be retained in the bladder, such as rhamnose. Alternatively, the desired effect can be established as a post-treatment reduction in sensitivity to potassium, as determined using the potassium sensitivity test, or as an improvement in the appearance and physiology of the bladder wall viewed cystoscopically or when examined histologically as biopsied tissue. Alternatively, the desired effect can be revealed as a reduction or easement in symptoms experienced by the patient.

Suitably, and in a preferred embodiment, the subject is treated by administering the drug by intravesical instillation, i.e., using a catheter to deliver the medicine directly into the bladder. For delivery into the bladder, HB-EGF is desirably provided in aqueous solution. It should be appreciated that there is a limit to the volume that a bladder can accommodate, and there is accordingly a limit to the volume of drug solution that can be administered to a given subject by instillation. For human bladders, such volume lies typically in the range from 10 mL to about 100 mL, more desirably in the range from 20 mL to about 75 mL, and suitably in the 40-60 mL range. At the upper end of the range (100 mL), the subject will have difficulty retaining the solution for the desired treatment period. At the lower end of the range (10 mL), the volume necessary to push sufficient drug through the catheter may not be reached. Most desirably, the volume of drug solution is at least sufficient to bathe/expose the entire bladder lining with drug.

In use, the drug solution is instilled and held by the patient for a period of at least about 30 minutes and desirably longer, before voiding. Repeated treatment, such as twice a week for 2-6 weeks followed by a reduced, maintenance regimen of once per week for a further 4-8 weeks may be performed. Treatment should be repeated until at least one of the symptoms subsides or resolves, such as pain, or urgency, or frequency. A reduction in these symptoms is usually measured empirically, using the Oleary Sant Index to establish scores at baseline and during treatment so that progress can be gauged.

For use, HB-EGF is thus desirably formulated as an aqueous solution, for example using saline or phosphate buffered saline (PBS) as vehicle. The HB-EGF is provided desirably in a unit dose per instillation that lies in the range from at least about 5 mgs to a maximum that is limited by the solubility of HB-EGF in the chosen vehicle. Suitably, HB-EGF is administered in a unit dose that is in the range from 10 mg to 1,000 mg per instillation. In embodiments, the unit dose of HB-EGF per instillation is 20 mg to 800 mgs, 30 mgs to 600 mgs, 40 mgs to 400 mgs, 50 mgs to 200 mgs, 75 mgs to 125 mgs, 90 mgs to 110 mgs, including 100 mgs. Thus, the HB-EGF can be formulated as a solution comprising these unit doses of HB-EGF in aqueous vehicle such as saline or PBS at a volume in the range from about 10 mL to 100 mL, from 20 mL to 50 mL, e.g., 15 mL, 20 mL, 25 mL. 30 mL, 35 mL, 40 mL, 45 mL or 50 mL. In a specific embodiment, the HB-EGF formulation comprises a unit dose of 100 mg HB-EGF in 20 mL PBS.

The present disclosure thus provides a composition comprising HB-EGF in a unit dose effective to treat cystitis and related conditions by intravesical instillation. It will be appreciated that such compositions also can be provided as multidose formulations, comprising 2 or more unit doses of HB-EGF, for subsequent dilution or fractionation prior to administration. In addition, the present disclosure also provides a kit, for use in the treatment of a subject presenting with a condition associated with damaged urothelium, the kit comprising;

(1) HB-EGF in a unit dose effective for such treatment,

(2) Optionally, an aqueous vehicle for reconstitution of the HB-EGF, and

(3) Instructions for the use thereof to treat the condition.

In the kit, the HB-EGF can be provided within any suitable, sterile container, such as a vial, ampoule or the like.

It will be appreciated that subjects treated with the HB-EGF composition can also be treated, in combination, with other drugs and agents useful to control the target disease and its symptoms. Other useful drugs include:
BCG (Bacillus Calmette-Guérin), originally a vaccine used to provide protection against tuberculosis, has been used for some time to treat different types of bladder cancer;
Chondroitin sulphate, which is available in different strengths under the brand names Uracyst® (2.0%) and Gepan® Instill (0.2%);
Corticosteroids can also be used intravesically, either alone or in a cocktail. Disodium cromoglycate is a substance that inhibits mast cells.
DMSO (dimethylsulfoxide)
Lidocaine (local anaesthetic) optionally with only sodium bicarbonate (to alkalize the lidocaine) or in combination with other drugs;
Pentosan polysulfate sodium;
Oxybutynin chloride;
Oxychlorosene sodium (Chlorpactin®), in a 0.2% concentration, for instance;

Sodium hyaluronate or hyaluronan, (also called hyaluronic acid),

The following non-limiting examples are illustrative of the present disclosure:

### EXAMPLES

In the examples that follow, the HB-EGF is human HB-EGF (74-148), a 75 residue soluble form of HB-EGF produced as a secreted recombinant protein in the yeast host, Pichia pastoris. Culture supernatant was harvested by centrifugation, filtered, and purified by cation-exchange chromatography followed by hydrophobic interaction chromatography. The resulting protein underwent ultrafiltration/diafiltration followed by ion-exchange chromatography. The final product was formulated in 150 mM sodium chloride, 20 mM sodium phosphate buffer, pH 6.0, and stored at 4° C.

### Example 1

HB-EGF Promotes Urothelial Cell Proliferation in Vivo

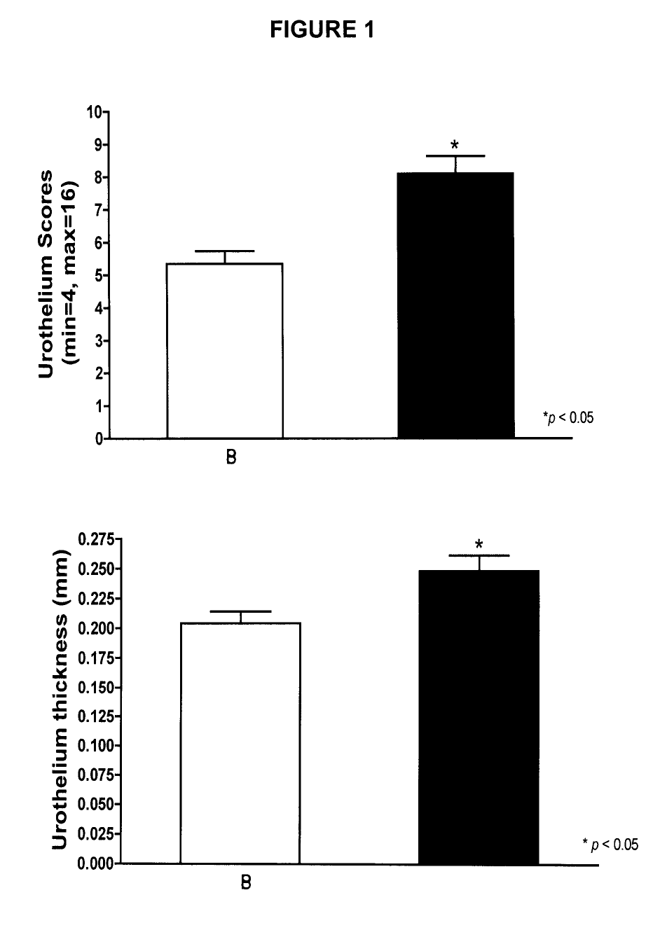

The effect of HB-EGF on urothelial cell proliferation in vivo was examined. Results are shown in FIG. 1. Female, 5-8 week old CBA/J mice were anesthetised with 2-3% isofluorane delivered by a vaporizer and catheterized with polyethylene catheter (I.D. 0.28 mm, O.D. 0.61 mm) attached to a 30GX½″ needle. HB-EGF(74-148) (10 mg/kg) or PBS was instilled (50 ul per bladder over 30 sec) via an infusion pump. Drug was retained in the bladder by occluding the urethras with collodion. After 2 hours, the collodion was removed and the anesthesia discontinued. Bladders were harvested 7 days later and 6 mm section (4 quadrants per bladder) stained with H&E and evaluated in a blinded fashion. (FIG. 1, Top) Urothelial proliferation was assessed using the following scoring system (max 16): 1=Normal−intact multi-layered urothelium, 2-3 cell layer thick, that sits on the sub-cellular matrix; 2=Marginal changes−minimal thickening of the urothelial cell layer, 3=Mild to moderate changes−mild, but clear hyperepithelialization; 4=Severe to extremely severe changes−gross hyper-epithelialized urothelium layer. (FIG. 1, Bottom) Urothelium thickness was measured with a stage micrometer (12 measurements per bladder). Data presented are pooled from 3 independent studies.

Thus, as shown in FIG. 1, intravesical administration of 10 mg/kg HB-EGF(74-148) to normal mice resulted in a detectable proliferation of bladder urothelium, as assessed by blinded tissue scoring and micrometer measurements. No effects were seen at doses less than or equal to 1 mg/kg in normal mice.

### Example 2

HB-EGF Reduced Permeability of Damaged Bladder Urothelium in Vivo

Five-week old female CBA/J mice were anesthetised with 2-3% isofluorane delivered by a vaporizer and catheterized with polyethylene catheter (I.D.0.28 mm, O.D. 0.61 mm) attached to a 30Gx½″ needle. Bladder damage was induced by exposure to 2% acetic acid (HAc) for 1 hr. Bladders were rinsed with PBS and mice rested for 0.5-1 hr. Mice then received daily treatments with HB-EGF (17.5 mg/kg) or PBS (2 hr retention).

HB-EGF was formulated in 150 mM sodium chloride, 20 mM sodium phosphate buffer, pH 6.0. It was delivered into the bladder by catheter (see above). Drug was retained in the bladder by occluding the urethras with collodion, which was removed after 2 hours.

Bladders were harvested at 48 hr post-acid treatment, and 6 mm sections (4 quadrants per bladder) stained with H&E and evaluated in a blinded fashion. Urothelial proliferation was assessed using the following scoring system (max 16): 0=hyper-re-epithelized urothelium; 1=Normal−intact multi-layered urothelium,2-3 cell layer thick, that sits on the sub-cellular matrix; 2=Mild injury−slight sloughing off of the superficial layer of urothelial cells.; 3=Moderate injury−loss of upper layer of urothelium; 4=Severe injury−complete denudation of urothelium or severely distorted parts of epithelial layer. 30 min before harvest, bladders were infused with 50 ul sodium fluorescein (NaF, 50 mg/ml). Blood was collected by cardiac puncture and plasma fluorescein detected in a de-glucuronidase assay.

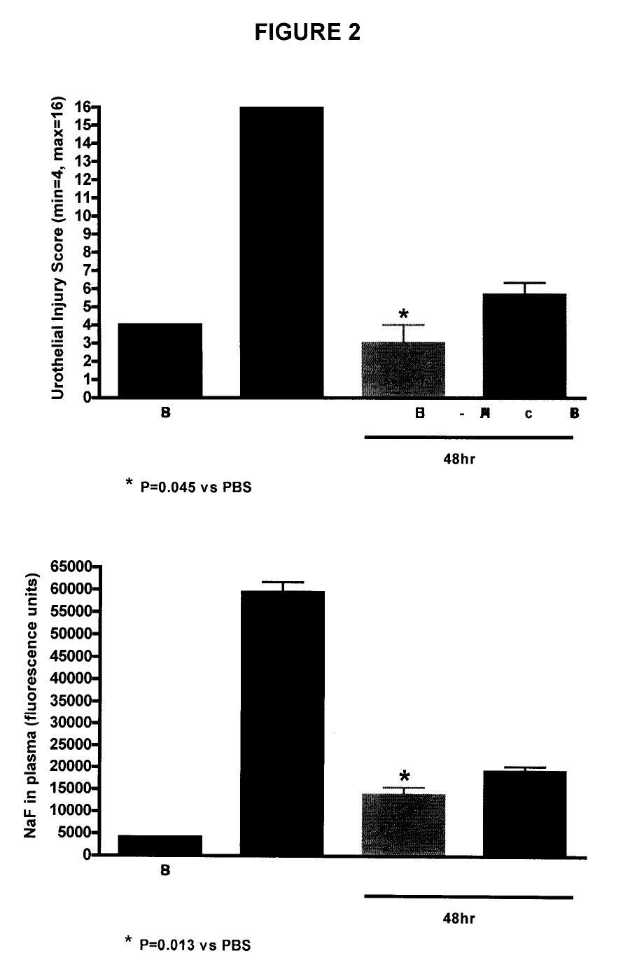

FIG. 2 summarizes data from 4 animal groups, as follows:
1) Control animals treated with PBS only (no acid damage)
2) Animals sacrificed 1 hr after acid damage
3) Animals exposed to acid damage and treated with two doses of HB-EGF, and sacrificed at 48 hours.
4) Animals exposed to acid damage and treated with two doses of PBS, and sacrificed at 48 hours.

In FIG. 2, the top graph shows a histological injury score. It reveals that acid treatment induces a high degree of damage at 1 hour, which is reduced by 48 hours in control (PBS) treated animals. This represents spontaneous re-epithelization (healing) of the bladder. Treatment with HB-EGF has an additional, statistically significant effect in promoting recovery.

The bottom panel of FIG. 2 reveals measures of in vivo permeability. Animals receive an intravesical instillation of sodium fluorescein, which produces a detectable fluorescence in plasma if the bladder is leaky. The data show a very large increase in permeability at 1 hour post-acid treatment, which is reduced by 48 hours in PBS treated animals. Treatment with HB-EGF has an additional, statistically significant effect in reducing permeability.

```
(human HB-EGF precursor)
SEQ ID NO: 1
    1 mkllpsvvlk lflaavlsal vtgeslerlr rglaagtsnp dpptvstdql lplgggrdrk
  61 vrdlqeadld llrvtlsskp qalatpnkee hgkrkkkgkg lgkkrdpclr kykdfcihge
121 ckyvkelrap scichpgyhg erchglslpv enrlytydht tilavvavvl ssvcllvivg
181 llmfryhrrg gydveneekv klgmtnsh
(human HB-EGF species)
SEQ ID NO: 2
vtlsskpqal atpnkeehgk rkkkgkglgk krdpclrkyk dfcihgecky vkelrapsci
chpgyhgerc hglsl
```

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

We claim:

1. A method for treating a subject having a condition associated with enhanced urothelium permeability, comprising the step of delivering by instillation to the urothelium of the subject a composition comprising HB-EGF in a unit dose effective to reduce urothelium permeability.

2. The method according to claim 1, wherein the condition is marked by sensitivity in the potassium sensitivity test.

3. The method according to claim 1, wherein the condition is interstitial cystitis.

4. The method according to claim 1, wherein the HB-EGF is human HB-EGF having SEQ ID NO:2.

5. The method according to claim 1, wherein the HB-EGF is delivered in a unit dose of from 50 mgs to 200 mgs.

6. The method according to claim 1, wherein said composition comprises an aqueous vehicle.

7. The method according to claim 6, wherein said composition has a volume of from 10 mL to 100 mL.

8. The method according to claim 1, wherein said composition comprises HB-EGF in a unit dose of from 50 to 200 mgs, and an aqueous vehicle having a volume of from 25-75 mL.

9. A pharmaceutical composition comprising HB-EGF in a unit dose effective to reduce permeability of urothelium in a subject in need thereof, and an aqueous vehicle having a volume in the range from 10 mL to 100 mL.

10. The pharmaceutical composition according to claim 9, wherein the aqueous vehicle is phosphate buffered saline.

11. The pharmaceutical composition according to claim 9, wherein the HB-EGF is human HB-EGF(74-148).

12. The pharmaceutical composition according to claim 10, comprising 50 mgs to 200 mgs of human HB-EGF(74-148) and an aqueous vehicle in a volume of from 25 mL to 75 mL.

13. A kit useful to treat a subject having a condition associated with aberrant urothelium permeability, comprising a unit dose of HB-EGF effective to reduce urothelium permeability upon instillation thereof to the bladder, and instructions for the use thereof to treat said condition.


SEQUENCE LISTING (2 sequences)

SEQ ID NO: 1 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 2 - protein, 75 aa, Homo sapiens
VTLSSKPQALATPNKEEHGKRKKKGKGLGKKRDPCLRKYKDFCIHGECKYVKELRAPSCICHPGYHGERCHGLSL